(19) United States
(12) Patent Application Publication
(10) Pub. No.: US 2020/0016163 A1
(43) Pub. Date: Jan. 16, 2020

(54) TREATMENT OF DEMENTIA WITH CANNABINOID AGONISTS

(71) Applicant: UNIVERSIDAD DEL PAIS VASCO/EUSKAL HERRIKO UNIBERTSITATEA, Leioa, Vizcaya (ES)

(72) Inventors: Rafael RODRIGUEZ PUERTAS, Leioa, Vizcaya (ES); Ivan MANUEL VICENTE, Leioa, Vizcaya (ES); Laura LOMBARDERO ITURRIZAGA, Leioa, Vizcaya (ES); Alberto LLORENTE OVEJERO, Leioa, Vizcaya (ES); Marta MORENO RODRIGUEZ, Leioa, Vizcaya (ES); Jonatan MARTINEZ GARDEAZABAL, Leioa, Vizcaya (ES); Estibaliz GONZALEZ DE SAN ROMAN MARTIN, Leioa, Vizcaya (ES)

(21) Appl. No.: 16/489,110

(22) PCT Filed: Feb. 23, 2018
(86) PCT No.: PCT/EP2018/054525

§ 371 Date: Aug. 27, 2019

(30) Foreign Application Priority Data
Mar. 2, 2017 (ES) P201730284

(51) Int. Cl. A61K 31/5383; A61K 9/00; A61P 25/28

(52) U.S. Cl. CPC A61K 31/5383; A61P 25/28; A61K 9/0019

(57) ABSTRACT

A medical treatment of irreversible-type dementias is provided, and more particularly to the use of a family of cannabinoid agonists to treat the impaired cognitive functions resulting from the dementias.

### FIELD OF THE INVENTION

The present invention is comprised in the field of medical treatment of the irreversible-type dementias, and it more particularly relates to the use of cannabinoid agonists for such purpose.

### BACKGROUND OF THE INVENTION

Dementia is characterized by a number of cognitive deficits involving a learning and/or memory impairment and at least one of the following cognitive disorders: aphasia, apraxia, agnosia or an executive function disorder. The subjects with dementia have an impaired ability to learn new information and forget material that was previously learned. The disorder is serious enough to significantly interfere in professional and social activities and may represent a deficit with respect to the subject's previous higher level of activity. For example, subjects with dementia can lose valuable objects, such as their wallet or keys, they forget the food they are cooking and can get lost in neighborhoods with which they are not familiar. In advanced forms, the memory impairment is so severe that the subject forgets his/her profession, level of schooling, anniversaries, family members, or sometimes even his/her own name.

The dementia can be reversible- or irreversible-type dementia. Reversible dementias are those dementias due to, for example, substance abuse, such as alcohol abuse, dietary deficiencies, such as a vitamin deficiency, head traumas, or fever or depression. In such cases, the dementia is normally reversed when the causing factor is eliminated.

Irreversible-type dementias, however, are related to specific clinical pictures (diseases) which entail impairment of the subject's cognitive ability, such as Alzheimer-type dementia, for example. In such cases, the cause of the mental impairment or brain damage cannot be reversed, but the subject's affected cognitive activity can be enhanced for example through the use of drugs. However, the scarce effectiveness of treatments that are currently used, such as cholinesterase (AChE) inhibitors like donepezil, rivastigmine and galantamine, as well as NMDA glutamate receptor blockers like memantine, is evident.

The prevalence of irreversible-type dementia is estimated to be almost 3% in adult populations, and up to 4% in 65 years and over populations, reaching a prevalence of 20% or higher in 85 years and over populations.

The high prevalence of irreversible-type dementias, as well as the enormous impact they have on the quality of life of those subjects suffering them and of the people on whom said subjects depend, entail a continuous and prevailing need to develop new treatments for irreversible-type dementias. On a molecular level there is certain controversy as to the effect produced by cannabinoid compounds on cognitive processes. There are some authors who support that cannabinoid compounds act as possible inhibitors of acetylcholine degradation enzymes (Gonzalez-Naranjo et al., Current Alzheimer Research 2013; 10:229-239), although the effect on said cognitive processes normally attributed to cannabinoid enhancers or agonists is a negative one. In this regard, Gessa et al. (Eur J Pharmacol 1998; 355(2-3):119-24) establish that the administration of certain cannabinoid compounds entails the drop in acetylcholine released in the prefrontal cortex and hippocampus; Marchalant et al. (Neuroscience, 2007; 144, 1516-1522; Neurobiology of aging, 2008; 29, 1894-1901) disclose spatial memory loss upon administration of the compound WIN55,212-2 in a model of brain inflammation. Furthermore, the controversy relating to the effect of cannabinoids also arises depending on the model of cognitive deficit used. For example, Ramirez et al., (The Journal of Neuroscience, 2005, 25(8):1904-1913), also based on another model of inflammation, which does not specifically represent the histopathological characteristics of Alzheimer's disease, and more specifically irreversible-type dementia, disclose that the administration of a cannabinoid appears to bring about improvements to the associated cognitive deficit.

Given the controversial observations from the state of the art, as well as the lack of studies in models of irreversible-type dementia, there is a prevailing need to identify treatments capable of counteracting the damage to the cortical-basal forebrain cholinergic pathway which occurs in patients suffering irreversible-type dementia and is what specifically controls memory and learning processes.

The authors of the present invention have now surprisingly discovered a group of cannabinoid agonists which, in a specific dose range, are useful in the treatment of irreversible-type dementias, specifically for enhancing the cognitive abilities affected as a result of said dementias.

### SUMMARY OF THE INVENTION

The main aspect of the present invention relates to the use of a cannabinoid agonist for the prevention or treatment of an irreversible-type dementia.

A cannabinoid agonist for use in the prevention or treatment of an irreversible-type dementia is also an object of the present invention.

The use of a cannabinoid agonist to prepare a medicinal product intended for the prevention or treatment of an irreversible-type dementia is also an object of the present invention.

A method of prevention or treatment of an irreversible-type dementia, which comprises: a) providing a subject in need of prevention or treatment of an irreversible-type dementia; and b) administering a cannabinoid agonist to said subject is also an object of the present invention.

### BRIEF DESCRIPTION OF THE DRAWINGS

FIG. 1 shows the time each experimental group (rats)spent in the target quadrant, where the escape hole is located. SHAM (needle injection); CSF (administration of CSF); SAP (administration of toxin 192 IgG-saporin); and CSF+WIN (administration of CSF+5 administrations of WIN55,212-2, 0.5 mg/kg i.p.) and SAP+WIN (administration of toxin 192 IgG-saporin+5 administrations of WIN55,212-2, 0.5 mg/kg i.p.).

FIG. 2 shows the time each experimental group spent in the target quadrant, where the escape hole is located. SAP* (administration of the toxin 192 IgG-saporin) and SAP*+WIN (administration of the toxin 192 IgG-saporin+5 administrations of WIN55,212-2, 0.5 mg/kg i.p.).

FIG. 3 shows the time each experimental group spent in the target quadrant, where the escape hole is located. SAP (administration of toxin 192 IgG-saporin), SAP+WIN (administration of toxin 192 IgG-saporin+5 administrations of WIN55,212-2, 0.5 mg/kg i.p.) and SAP+W+SR (administration of toxin 192 IgG-saporin+5 administrations of WIN55,212-2, 0.5 mg/kg, and SR141716A, 0.5 mg/kg i.p.).

FIG. 4 shows the time each experimental group spent in the target quadrant, where the escape hole is located. SAP (administration of toxin 192 IgG-saporin), CSF (administration of CSF) and SAP+W3 (administration of toxin 192 IgG-saporin+5 administrations of WIN55,212-2, at 3 mg/kg and i.p.) and CSF+W3 (administration of CSF+5 administrations of WIN55,212-2, at 3 mg/kg i.p.).

### DETAILED DESCRIPTION OF THE INVENTION

The term “cannabinoid agonist” refers to a compound that binds to one or more cannabinoid receptors to exercise an agonist effect or partial agonist effect.

In one embodiment, the cannabinoid agonist is selected from the group consisting of:

compounds of formula (Ia) or (Ib)

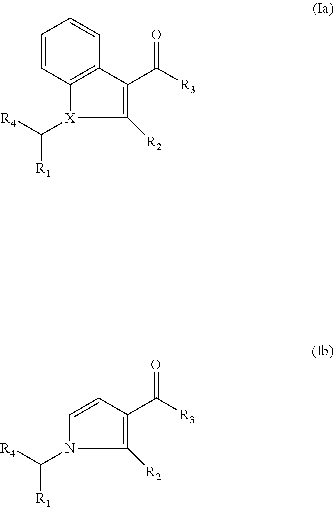

wherein
X is N or CH;
R₁ is selected from
   C₂-C₆ alkyl;
   a piperidinyl, pyrrolidinyl or oxazinanyl group that is unsubstituted or substituted with methyl at its nitrogen atom; and
   a —CH₂-morpholinyl, —CH₂-thiomorpholinyl or —CH₂— piperidinyl group;
R₂ is selected from H, C₁-C₆ alkyl, halogen, —CN and —OH;
R₃ is selected from unsubstituted aryl and aryl substituted at one or more of its available positions with C₁-C₆ alkyl, —O—C₁-C₆ alkyl, halogen, —CN, —NO₂ or —OH. The substitutions in each available position are understood to be independent of one another, where the aryl may therefore have substituents of different types;
R₄ is H, and in the compound of formula (Ia) it can also be a —CH₂—Y group, wherein Y is O, S, or NR₅, wherein Y is bound directly to the 7-position of the indole ring (X is N) or indene ring (X is CH), and wherein R₅ is H or C₁-C₆ alkyl;

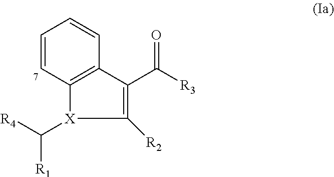

and wherein in the compound of formula (Ia), the phenyl portion of the indole ring (X is N) or indene ring (X is CH) is unsubstituted or substituted at one or more of its available positions with C₁-C₆ alkyl, —O—C₁-C₆ alkyl or halogen. The substitutions in each available position are understood to be independent of one another, where the phenyl portion may therefore have substituents of different types;

or a salt or solvate thereof;

the following other synthetic CB₁ agonists:

| | | Disclosure |
|---|---|---|
| Name | IUPAC Name | in Ref. |
| Pravadoline | (4-Methoxyphenyl){2-methyl-1- | [1], [2] |
| (WIN 48,098) | [2-(4-morpholinyl)ethyl]-1H- | |
| | indol-3-yl}methanone | |
| JWH-007 | 1-Pentyl-2-methyl-3-(1- | [1], [2] |
| | naphthoyl)indole | |
| JWH-015 | (2-Methyl-1-propyl-1H-indol-3- | [1], [2], |
| | yl)-1-naphthalenylmethanone | [8] |
| JWH-016 | (1-Butyl-2-methyl-1H-indol-3- | [1], [2] |
| | yl)-1-naphthalenyl-methanone | |
| JWH-018 | Naphthalen-1-yl-(1- | [1], [2], |
| (AM-678) | pentylindol-3-yl)methanone | [8] |
| JWH-019 | 1-Hexyl-3-(naphthalen-1- | [1], [2] |
| | oil)indole | |
| JWH-030 | 1-Pentyl-3-(1- | [1], [2] |
| | naphthoyl)pyrrole | |
| JWH-047 | (1-Butyl-2-methyl-1H-indol-3- | [1], [2] |
| | yl)(7-methyl-1- | |
| | naphthalenyl)methanone | |
| JWH-048 | (1-Pentyl-2-methyl-1H-indol-3- | [1], [2] |
| | yl)(7-methyl-1- | |
| | naphthalenyl)methanone | |
| JWH-051 | ((6aR,10aR)-6,6-Dimethyl-3-(2- | [1], [2] |
| | methyloctan-2-yl)-6a,7,10,10a- | |
| | tetrahydrobenzo[c]chromen-9- | |
| | yl)methanol | |
| JWH-073 | Naphthalen-1-yl-(1-butylindol- | [1], [2] |
| | 3-yl)methanone | |
| JWH-081 | 4-Methoxynaphthalen-1-yl-(1- | [1], [2] |
| | pentylindol-3-yl)methanone | |
| JWH-098 | 4-Methoxynaphthalen-1-yl-(1- | [1], [2] |
| | pentyl-2-methylindol-3- | |
| | yl)methanone | |
| JWH-120 | (4-Methyl-1-naphthalenyl)(1- | [1], [2] |
| | propyl-1H-indol-3-yl)methanone | |
| JWH-122 | (4-Methyl-1-naphthyl)-(1- | [1], [2] |
| | pentylindol-3-yl)methanone | |
| JWH-147 | (1-Hexyl-5-phenyl-1H-pyrrol-3- | [1], [2] |
| | yl)-1-naphthalenyl-methanone | |
| JWH-148 | (4-Methyl-1-naphthalenyl)(2- | [1], [2] |
| | methyl-1-propyl-1H-indol-3- | |
| | yl)methanone | |
| JWH-149 | (4-Methyl-1-naphthalenyl)(2- | [1], [2] |
| | methyl-1-pentyl-1H-indol-3- | |
| | yl)methanone | |
| JWH-164 | 7-Methoxynaphthalen-1-yl-(1- | [1], [2] |
| | pentylindol-3-yl)methanone | |
| JWH-167 | 2-Phenyl-1-(1-pentylindol-3- | [1], [2] |
| | yl)ethanone | |
| JWH-175 | (1-Pentylindol-3- | [1], [2] |
| | yl)naphthalen-1-ylmethane | |
| JWH-176 | 1-([(1E)-3-Pentylinden-1- | [1], [2] |
| | ylidine]methyl)naphthalene | |
| JWH-181 | (2-Methyl-1-pentyl-1H-indol-3- | [1], [2] |
| | yl)(4-propyl-1- | |
| | naphthalenyl)methanone | |
| JWH-182 | 4-Ethylnaphthalen-1-yl-(1- | [1], [2] |
| | pentylindol-3-yl)methanone | |
| JWH-184 | 3-[(4-Methyl-1- | [1], [2] |
| | naphthalenyl)methyl]-1-pentyl- | |
| | 1H-indole | |
| JWH-185 | 3-[(4-Methoxy-1- | [1], [2] |
| | naphthalenyl)methyl]-1-pentyl- | |
| | 1H-indole | |
| JWH-192 | (1-(2-Morpholin-4- | [1], [2] |
| | ylethyl)indol-3-yl)-4- | |
| | methylnaphthalen-1-ylmethane | |
| JWH-193 | (1-(2-Morpholin-4- | [1], [2] |
| | ylethyl)indol-3-yl)-4- | |
| | methylnaphthalen-1-ylmethanone | |
| JWH-194 | 2-Methyl-1-pentyl-1H-indol-3- | [1], [2] |
| | yl-(4-methyl-1- | |
| | naphthyl)methane | |
| JWH-195 | (1-(2-Morpholin-4- | [1], [2] |
| | ylethyl)indol-3-yl)- | |
| | naphthalen-1-ylmethane | |
| JWH-196 | 2-Methyl-3-(1- | [1], [2] |
| | naphthalenylmethyl)-1-pentyl- | |
| | 1H-Indole | |
| JWH-197 | 2-Methyl-1-pentyl-1H-indol-3- | [1], [2] |
| | yl-(4-methoxy-1- | |
| | naphthyl)methane | |
| JWH-198 | (1-(2-Morpholin-4- | [1], [2] |
| | ylethyl)indol-3-yl)-4- | |
| | methoxynaphthalen-1- | |
| | ylmethanone | |
| JWH-199 | (1-(2-Morpholin-4- | [1], [2] |
| | ylethyl)indol-3-yl)-4- | |
| | methoxynaphthalen-1-ylmethane | |
| JWH-200 | (1-(2-Morpholin-4- | [1], [2] |
| | ylethyl)indol-3-yl)- | |
| | naphthalen-1-ylmethanone | |
| JWH-201 | 2-(4-Methoxyphenyl)-1-(1- | [1], [2] |
| | pentyl-1H-indol-3-yl)-ethanone | |
| JWH-202 | l-Pentyl-2-methyl-3-(4- | [1], [2] |
| | methoxyphenylacetyl)indole | |
| JWH-203 | 2-(2-Chlorophenyl)-1-(1- | [1], [2] |
| | pentylindol-3-yl)ethanone | |
| JWH-204 | 2-(2-Chlorophenyl)-1-(2- | [1], [2] |
| | methyl-1-pentyl-1H-indol-3- | |
| | yl)ethanone | |
| JWH-205 | 1-(2-Methyl-1-pentyl-1H-indol- | [1], [2] |
| | 3-yl)-2-phenylethanone | |
| JWH-206 | 2-(4-Chlorophenyl)-1-(1- | [1], [2] |
| | pentyl-1H-indol-3-yl)ethanone | |
| JWH-207 | 2-(4-Chlorophenyl)-1-(2- | [1], [2] |
| | methyl-1-pentyl-1H-indol-3- | |
| | yl)ethanone | |
| JWH-208 | 2-(4-Methylphenyl)-1-(1- | [1], [2] |
| | pentyl-1H-indol-3-yl)ethanone | |
| JWH-209 | 1-(2-Methyl-1-pentyl-1H-indol- | [1], [2] |
| | 3-yl)-2-(4- | |
| | methylphenyl)ethanone | |
| JWH-210 | 4-Ethylnaphthalen-1-yl-(1- | [1], [2] |
| | pentylindol-3-yl)methanone | |
| JWH-211 | (4-Ethyl-1-naphthyl)(2-methyl- | [1], [2] |
| | 1-propyl-1H-indol-3- | |
| | yl)methanone | |
| JWH-212 | (4-Ethyl-1-naphthyl)(1-propyl- | [1], [2] |
| | 1H-indol-3-yl)methanone | |
| JWH-213 | (4-Ethyl-1-naphthyl)(2-methyl- | [1], [2] |
| | 1-pentyl-1H-indol-3- | |
| | yl)methanone | |
| JWH-234 | (7-Ethyl-1-naphthyl)(1-pentyl- | [1], [2] |
| | 1H-indol-3-yl)methanone | |
| JWH-235 | (7-Ethyl-1-naphthyl)(1-propyl- | [1], [2] |
| | 1H-indol-3-yl)methanone | |
| JWH-236 | (7-Ethyl-1-naphthyl)(2-methyl- | [1], [2] |
| | 1-propyl-1H-indol-3- | |
| | yl)methanone | |
| JWH-237 | 2-(3-Chlorophenyl)-1-(1- | [1], [2] |
| | pentyl-1H-indol-3-yl)ethanone | |
| JWH-239 | (4-Butyl-1-naphthyl)(1-propyl- | [1], [2] |
| | 1H-indol-3-yl)methanone | |
| JWH-240 | (4-Butyl-1-naphthyl)(1-pentyl- | [1], [2] |
| | 1H-indol-3-yl)methanone | |
| JWH-241 | (4-Butyl-1-naphthyl)(2-methyl- | [1], [2] |
| | 1-propyl-1H-indol-3- | |
| | yl)methanone | |
| JWH-242 | (4-Butyl-1-naphthyl)(2-methyl- | [1], [2] |
| | 1-pentyl-1H-indol-3- | |
| | yl)methanone | |
| JWH-243 | [5-(4-Methoxyphenyl)-1-pentyl- | [1], [2] |
| | 1H-pyrrol-3-yl](1- | |
| | naphthyl)methanone | |
| JWH-244 | [5-(4-Methylphenyl)-1-pentyl- | [1], [2] |
| | 1H-pyrrol-3-yl](1- | |
| | naphthyl)methanone | |
| JWH-245 | [5-(4-Chlorophenyl)-1-pentyl- | [1], [2] |
| | 1H-pyrrol-3-yl](1- | |
| | naphthyl)methanone | |
| JWH-246 | [5-(3-Chlorophenyl)-1-pentyl- | [1], [2] |
| | 1H-pyrrol-3-yl](1- | |
| | naphthyl)methanone | |
| JWH-248 | 2-(4-Bromophenyl)-1-(1-pentyl- | [1], [2] |
| | 1H-indol-3-yl)ethanone | |
| JWH-249 | (1-Pentyl-3-(2- | [1], [2] |
| | bromophenylacetyl)indole) | |
| JWH-250 | 2-(2-Methoxyphenyl)-1-(1- | [1], [2] |
| | pentylindol-3-yl)ethanone | |
| JWH-251 | 2-(2-Methylphenyl)-1-(1- | [1], [2] |
| | pentyl-1H-indol-3-yl)ethanone | |
| JWH-252 | 1-(2-Methyl-1-pentyl-1H-indol- | [1], [2] |
| | 3-yl)-2-(2- | |
| | methylphenyl)ethenone | |
| JWH-253 | 2-(3-Methoxyphenyl)-1-(2- | [1], [2] |
| | methyl-1-pentyl-1H-indol-3- | |
| | yl)ethenone | |
| JWH-258 | (4-Ethoxy-1-naphthyl) (1- | [1], [2] |
| | pentyl-1H-indol-3-yl)methanone | |
| JWH-259 | (4-Ethoxy-1-naphthyl) (1- | [1], [2] |
| | propyl-1H-indol-3-yl)methanone | |
| JWH-260 | (4-Ethoxy-l-naphthyl)(2- | [1], [2] |
| | methyl-1-pentyl-1H-indol-3- | |
| | yl)methanone | |
| JWH-261 | (4-Ethoxy-1-naphthyl)(2- | [1], [2] |
| | methyl-1-propyl-1H-indol-3- | |
| | yl)methanone | |
| JWH-262 | (7-Ethyl-1-naphthyl)(2-methyl- | [1], [2] |
| | 1-pentyl-1H-indol-3- | |
| | yl)methanone | |
| JWH-265 | (2-Methoxy-1-naphthyl)(1- | [1], [2] |
| | propyl-1H-indol-3-yl)methanone | |
| JWH-267 | (2-Methoxy-1-naphthyl)(1- | [1], [2] |
| | pentyl-1H-indol-3-yl)methanone | |
| JWH-268 | (2-Methoxy-1-naphthyl)(2- | [1], [2] |
| | methyl-1-pentyl-1H-indol-3- | |
| | yl)methanone | |
| JWH-292 | [5-(2-Methoxyphenyl)-1-pentyl- | [1], [2] |
| | lH-pyrrol-3-yl](1- | |
| | naphthyl)methanone | |
| JWH-293 | 1-Naphthyl[5-(3-nitrophenyl)- | [1], [2] |
| | 1-pentyl-1H-pyrrol-3- | |
| | yl]methanone | |
| JWH-302 | 2-(3-Methoxyphenyl)-1-(1- | [1], [2] |
| | pentyl-1H-indol-3-yl)ethanone | |
| JWH-303 | 2-(3-Chlorophenyl)-1-(2- | [1], [2] |
| | methyl-1-pentyl-1H-indol-3- | |
| | yl)ethanone | |
| JWH-305 | 2-(2-Bromophenyl)-1-(2-methyl- | [1], [2] |
| | 1-pentyl-1H-indol-3- | |
| | yl)ethanone | |
| JWH-306 | 2-(2-Methoxyphenyl)-1-(2- | [1], [2] |
| | methyl-1-pentyl-1H-indol-3- | |
| | yl)ethanone | |
| JWH-307 | [5-(2-Fluorophenyl)-1-pentyl- | [1], [2], |
| | 1H-pyrrol-3-yl](1- | [9] |
| | naphthyl)methanone | |
| JWH-308 | [5-(4-Fluorophenyl)-1-pentyl- | [1], [2], |
| | 1H-pyrrol-3-yl](1- | [9] |
| | naphthyl)methanone | |
| JWH-311 | 2-(2-Fluorophenyl)-1-(1- | [1], [2] |
| | pentyl-1H-indol-3-yl)ethanone | |
| JWH-312 | 2-(3-Fluorophenyl)-1-(1- | [1], [2] |
| | pentyl-1H-indol-3-yl)ethanone | |
| JWH-313 | 2-(4-Fluorophenyl)-1-(1- | [1], [2] |
| | pentyl-1H-indol-3-yl)ethanone | |
| JWH-314 | 2-(2-Fluorophenyl)-1-(2- | [1], [2] |
| | methyl-1-pentyl-1H-indol-3- | |
| | yl)ethanone | |
| JWH-315 | 2-(3-Fluorophenyl)-1-(2- | [1], [2] |
| | methyl-1-pentyl-1H-indol-3- | |
| | yl)ethenone | |
| JWH-346 | [5-(3-Methylphenyl)-1-pentyl- | [1], [2], |
| | 1H-pyrrol-3-yl](1- | [9] |
| | naphthyl)methanone | |
| JWH-348 | 1-Naphthyl{l-pentyl-5-[4- | [1], [2] |
| | (trifluoromethyl)phenyl]-1H- | |
| | pyrrol-3-yl}methanone | |
| JWH-359 | (6aR,10aR)-3-[(3R)-2,3- | [1], [2] |
| | Dimethyl-2-pentanyl]-1- | |
| | methoxy-6,6,9-trimethyl- | |
| | 6a,7,10,10a-tetrahydro-6H- | |
| | benzo[c]chromene | |
| JWH-363 | 1-Naphthyl{1-pentyl-5-[3- | [1], [2], |
| | (trifluoromethyl)phenyl]-1H- | [9] |
| | pyrrol-3-yl}methanone | |
| JWH-364 | [5-(4-Ethylphenyl)-1-pentyl- | [1], [2], |
| | 1H-pyrrol-3-yl](1- | [9] |
| | naphthyl)methanone | |
| JWH-365 | [5-(2-Ethylphenyl)-1-pentyl- | [1], [2], |
| | 1H-pyrrol-3-yl](1- | [9] |
| | naphthyl)methanone | |
| JWH-367 | [5-(3-Methoxyphenyl)-1-pentyl- | [1], [2], |
| | 1H-pyrrol-3-yl](1- | [9] |
| | naphthyl)methanone | |
| JWH-368 | [5-(3-Fluorophenyl)-1-pentyl- | [1], [2] |
| | 1H-pyrrol-3-yl](1- | |
| | naphthyl)methanone | |
| JWH-369 | [5-(2-Chlorophenyl)-1-pentyl- | [1], [2] |
| | 1H-pyrrol-3-yl](1- | |
| | naphthyl)methanone | |
| JWH-370 | [5-(2-Methylphenyl)-1-pentyl- | [1], [2], |
| | 1H-pyrrol-3-yl](1- | [9] |
| | naphthyl)methanone | |
| JWH-371 | [5-(4-Butylphenyl)-1-pentyl- | [1], [2], |
| | 1H-pyrrol-3-yl](1- | [9] |
| | naphthyl)methanone | |
| JWH-373 | [5-(2-Butylphenyl)-1-pentyl- | [1], [2] |
| | 1H-pyrrol-3-yl](1- | |
| | naphthyl)methanone | |
| JWH-387 | (4-Bromo-1-naphthyl)(1-pentyl- | [1], [2], |
| | 1H-indol-3-yl)methanone | [8] |
| JWH-392 | (1R,3R,4R)-4-(3- | [1], [2] |
| | Hydroxypropyl)-3-[4-(2-methyl- | |
| | 2-pentanyl)phenyl]cyclohexanol | |
| JWH-394 | 2-Methyl-N-pentyl-3-(4-bromo- | [1], [2], |
| | 1-naphthoyl) | [8] |
| JWH-395 | 2-Methyl-N-propyl-3-(4-bromo- | [1], [2], |
| | 1-naphthoyl)indole | [8] |
| JWH-397 | 2-Methyl-N-pentyl-3-(4-chloro- | [1], [2], |
| | 1-naphthoyl)indole | [8] |
| JWH-398 | (4-Chloro-1-naphthyl)(1- | [1], [2], |
| | pentyl-1H-indol-3-yl)methanone | [8] |
| JWH-399 | 2-Methyl-N-propyl-3-(4-chloro- | [1], [2], |
| | 1-naphthoyl)indole | [8] |
| JWH-400 | N-Propyl-3-(4-chloro-1- | [1], [2], |
| | naphthoyl)indole | [8] |
| JWH-412 | (4-Fluoro-1-naphthyl)(1- | [1], [2], |
| | pentyl-1H-indol-3-yl)methanone | [8] |
| JWH-413 | 2-Methyl-N-pentyl-3-(4-fluoro- | [1], [2], |
| | 1-naphthoyl)indole | [8] |
| JWH-414 | N-propyl-3-(4-fluoro-1- | [1], [2], |
| | naphthoyl)indole | [8] |
| JWH-415 | 2-Methyl-N-propyl-3-(4-fluoro- | [1], [2], |
| | 1-naphthoyl)indole | [8] |
| AM-087 | (6aR,10aR)-3-(6-Bromo-2- | [2] |
| | methyl-2-hexanyl)-6,6,9- | |
| | trimethyl-6a,7,10,10a- | |
| | tetrahydro-6H-benzo[c]chromen- | |
| | 1-ol | |
| AM-411 | (6aR,10aR)-3-(Adamantan-1-yl)- | [2] |
| | 6,6,9-trimethyl-6a,7,10,10a- | |
| | tetrahydro-6H-benzo[c]chromen- | |
| | 1-ol | |
| AM-679 | 1-Pentyl-3-(2- | [2] |
| | iodobenzoyl)indole | |
| AM-694 | [1-(5-Fluoropentyl)-1H-indol- | [2] |
| | 3-yl](2-iodophenyl)methanone | |
| AM-905 | (6aR,9R,10aR)-3-[(1E)-1- | [45] |
| | Hepten-1-yl]-9- | |
| | (hydroxymethyl)-6,6-dimethyl- | |
| | 6a,7,8,9,10,10a-hexahydro-6H- | |
| | benzo[c]chromen-1-ol | |
| AM-906 | (6aR,9R,10aR)-3-[(1Z)-1- | [4] |
| | Hepten-1-yl]-9- | |
| | (hydroxymethyl)-6,6-dimethyl- | |
| | 6a,7,8,9,10,10a-hexahydro-6H- | |
| | benzo[c]chromen-1-ol | |
| AM-919 | (6aR,9R,10aR)-9- | [5] |
| | (Hydroxymethyl)-6-(3- | |
| | hydroxypropyl)-6-methyl-3-(2- | |
| | methyloctan-2-yl)- | |
| | 6a,7,8,9,10,10a-hexahydro-6H- | |
| | benzo[c]chromen-1-ol | |
| AM-938 | (6R,6aR,9R,10aR)-9- | [5] |
| | (Hydroxymethyl)-6-(3-hydroxy- | |
| | 1-propyn-1-yl)-6-methyl-3-(2- | |
| | methyl-2-octanyl)- | |
| | 6a,7,8,9,10,10a-hexahydro-6H- | |
| | benzo[c]chromen-1-ol | |
| AM-1220 | (1-{[(2R)-1-Methyl-2- | [6] |
| | piperidinyl]methyl}-1H-indol- | |
| | 3-yl) (1-naphthyl)methanone | |
| AM-2201 | [1-(5-Fluoropentyl)-1H-indol- | [7] |
| | 3-yl](1-naphthyl)methanone | |
| AM-2232 | 5-[3-(1-Naphthoyl)-1H-indol-1- | [10] |
| | yl]pentanenitrile | |
| AM-2233 | (2-Iodophenyl){1-[(1-methyl-2- | [11] |
| | piperidinyl)methyl]-1H-indol- | |
| | 3-yl}methanone | |
| AM-2389 | (6aR,9R,10aR)-3-(1- | [12] |
| | Hexylcyclobutyl)-6,6-dimethyl- | |
| | 6a,7,8,9,10,10a-hexahydro-6H- | |
| | benzo[c]chromene-1,9-diol | |
| AM-4030 | (6S,6aR,9R,10aR)-9- | [13] |
| | (Hydroxymethyl)-6-[(1E)-3- | |
| | hydroxy-1-propen-1-yl]-6- | |
| | methyl-3-(2-methyl-2-octanyl)- | |
| | 6a,7,8,9,10,10a-hexahydro-6H- | |
| | benzo[c]chromen-1-ol | |
| APICA | N-[(3s,5s,7s)-Adamantan-1-yl]- | [14] |
| (SBD-001) | 1-pentyl-1H-indole-3- | |
| | carboxamide | |
| CP55940 | 2-[(1R,2R,5R)-5-hydroxy-2-(3- | [1], [2] |
| | hydroxypropyl)cyclohexyl]-5- | |
| | (2-methyloctan-2-yl)phenol | |
| Otenabant | 1-[8-(2-chlorophenyl)-9-(4- | [1] |
| (CP-945,598) | chlorophenyl)purin-6-yl]-4- | |
| | ethylaminopiperidine-4- | |
| | carboxamide | |
| Nabilone | 1-hydroxy-6,6-dimethyl-3-(2- | [1] |
| (Cesamet ®) | methyloctan-2-yl)-7,8,10,10a- | |
| | tetrahydro-6aH- | |
| | benzo[c]chromen-9-one | |
| Δ⁹- | (6aR,10aR)-6,6,9-trimethyl-3- | [1], [2] |
| tetrahydro- | pentyl-6a,7,8,10a- | |
| cannabinol | tetrahydrobenzo[c]chromen-1-ol | |
| (Marinol ®) | | |
| Δ 8- | 6,6,9-trimethyl-3-pentyl- | [1], [2] |
| tetrahydro- | 6a,7,10,10a- | |
| cannabinol | tetrahydrobenzo[c]chromen-1-ol | |
| Cannabinol | 6,6,9-trimethyl-3- | [1] |
| | pentylbenzo[c]chromen-1-ol | |
| HU-210 | (6aR,10aR)-9-(hydroxymethyl)- | [1], [2] |
| | 6,6-dimethyl-3-(2-methyloctan- | |
| | 2-yl)-6a,7,10,10a- | |
| | tetrahydrobenzo[c]chromen-l-ol | |

and

the following endocannabinoids:

| Name | IUPAC Name | Ref. |
|---|---|---|
| Anandamide (AEA) | (5Z,8Z,11Z,14Z)-N-(2- | [1], [2] |
| | Hydroxyethyl)-5,8,11,14- | |
| | icosatetraenamide | |
| 2- | 1,3-Dihydroxy-2-propanyl | [1], [2] |
| Arachidonoylglycerol | (5Z,8Z,11Z,14Z)-5,8,11,14- | |
| (2-AG) | icosatetraenoate | |
| N- | (7Z,10Z,13Z,16Z)-N-(2- | [1], [2] |
| Docosatetraenoyl- | Hydroxyethyl)-7,10,13,16- | |
| ethanolamine | docosatetraenamide | |
| N-dihomo-y- | (8Z,11Z,14Z)-N-(2- | [1], [2] |
| linolenoylethanolamine | hydroxyethyl)icosa-8,11,14- | |
| | trienamide | |
| 2-Arachidonyl | 2-[(5Z,8Z,11Z,14Z)-5,8,11,14- | [1], [2] |
| glyceryl ether | Icosatetraen-1-yloxy]-1,3- | |
| (Noladin ether) | propanediol | |
| Virodhamine | 2-Aminoethyl (5Z,8Z,11Z,14Z)- | [2] |
| | 5,8,11,14-icosatetraenoate | |
| N- | N-(2- | [2] |
| Palmitoylethanolamine | Hydroxyethyl)hexadecanamide | |
| (PEA) | | |
| Oleamide (ODA) | (9Z)-9-Octadecenamida | [2] |
| N- | (5Z,8Z,11Z,14Z)-N-[2-(3,4- | [2] |
| Arachidonoyldopamine | Dihydroxyphenyl)ethyl]- | |
| | 5,8,11,14-icosatetraenamide | |
| N- | N-(2- | [2] |
| Stearoylethanolamine | Hydroxyethyl)octadecanamide | |
| (SEA) | | |
| N-Oleoylethanolamine | (9E)-N-(2-Hydroxyethyl)-9- | [2] |
| (OEA) | octadecenamide | |
| N- | (2E,4E,6E,8E,10E,12E)-N-(2- | [2] |
| Docosahexaenoyl- | Hydroxyethyl)-2,4,6,8,10,12- | |
| ethanolamine (DHEA) | docosahexaenamide | |
| N- | (5Z,8Z,11Z,14Z)-N- | [2] |
| Arachidonoylcyclo- | cyclopropylicosa-5,8,11,14- | |
| propylamine (ACPA) | tetraenamide | |
| Arachidonyl-2- | (5Z,8Z,11Z,14Z)-N-(2- | [2] |
| chloroethylamide | chloroethyl)icosa-5,8,11,14- | |
| (ACEA) | tetraenamida | |
| O-1812 | (5Z,8Z,11Z,14Z)-20-cyano-N- | [2] |
| | [(2R)-1-Hydroxypropan-2-yl]- | |
| | 16,16-dimethylicosa-5,8,11,14- | |
| | tetraenamida | |

In a preferred embodiment, the cannabinoid agonist is a compound of formula (Ia) or (Ib) as defined above, or a salt or solvate thereof. In a particularly preferred embodiment, it is a compound of formula (Ia).

In a preferred embodiment, X is N.

In one embodiment, R₁ is a piperidinyl, pyrrolidinyl or oxazinanyl group, and more preferably it is a 2-piperidinyl, 2-pyrrolidinyl or 4-oxazinanyl group, as depicted below:

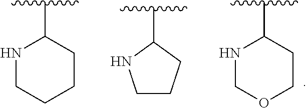 [SMILES: *C1CCCCN1 ; *C1CCCN1 ; *C1CCOCN1]

In a more particular embodiment, the nitrogen atom of the piperidinyl, pyrrolidinyl and oxazinanyl rings is substituted with methyl.

In a preferred embodiment, R₁ is a —CH₂-morpholinyl, —CH₂— thiomorpholinyl or —CH₂-piperidinyl group, and more preferably it is a —CH₂-(4-morpholinyl), —CH₂-(4-thiomorpholinyl) or —CH₂-(1-piperidinyl) group, as depicted below:

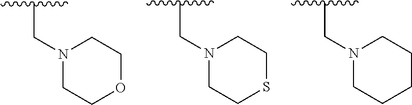 [SMILES: *CN1CCOCC1 ; *CN1CCSCC1 ; *CN1CCCCC1]

Even more preferably, R₁ is a —CH₂-morpholinyl group, preferably —CH₂-(4-morpholinyl).

In a preferred embodiment, R₂ is H or C₁-C₆ alkyl, preferably H or methyl, even more preferably methyl.

In a preferred embodiment, R₃ is phenyl or naphthyl. Preferably it is naphthyl, even more preferably 1-naphthyl.

In a preferred embodiment, R₃ is unsubstituted aryl. In a preferred embodiment, R₃ is unsubstituted phenyl or unsubstituted naphthyl. Preferably it is unsubstituted naphthyl, even more preferably unsubstituted 1-naphthyl.

In a preferred embodiment, R₄ in the compound of formula (Ia) is a —CH₂—Y group, wherein Y is O, S, or NR₅, wherein Y is bound directly to the 7-position of the indole ring (X is N) or indene ring (X is CH), and wherein R₅ is H or C₁-C₆ alkyl. In other words, compound (Ia) is a compound of formula:

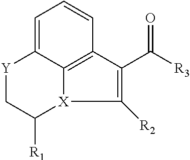

wherein R₁, R₂, R₃, X e Y are as defined in any embodiment described herein.

In an even more preferred embodiment, R₄ in the compound of formula (Ia) is a —CH₂—Y group, wherein Y is O and is bound directly to the 7-position of the indole ring (X is N) or indene ring (X is CH).

In a preferred embodiment, in the compound of formula (Ia) the phenyl portion of the indole ring (X is N) or indene ring (X is CH) is unsubstituted.

The preceding structural embodiments may be combined freely with one another to generate new, more specific embodiments.

In a preferred particular embodiment, the cannabinoid agonist is

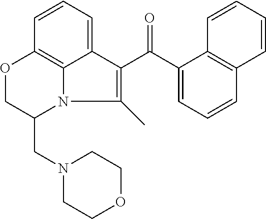 [SMILES: Cc1c(C(=O)c2cccc3ccccc23)c2cccc3c2n1C(CN1CCOCC1)CO3]

also known as WIN 55212, or a salt or solvate thereof. Preferably, the cannabinoid agonist is WIN 55212.

In a particularly preferred particular embodiment, the cannabinoid agonist is

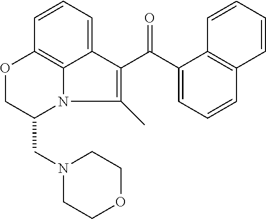 [SMILES: Cc1c(C(=O)c2cccc3ccccc23)c2cccc3c2n1[C@H](CN1CCOCC1)CO3]

also known as WIN 55212-2, or a salt or solvate thereof. Preferably, the cannabinoid agonist is WIN 55212-2.

The aforementioned cannabinoid agonists can be obtained from commercial sources or be prepared by chemical synthesis methods described in the state of the art, or variations thereof that fall within the common knowledge of one skilled in the art. The document by D'Ambra et al. (J. Med. Chem. 1992, 35, 124-135) describes the synthesis of the compounds of formula (Ia) or (Ib). On the other hand, compound WIN 55212-2, for example, is marketed, among many others, by Sigma-Aldrich in mesylate salt form (CAS no.: 131543-23-2), Tocris Bioscience (Ref. 1038), Adooq Bioscience (Ref. A11932), or MedChem Express (Ref. HY-13291).

“Alkyl” refers to a radical having a straight or branched hydrocarbon chain that consists of the number of carbons indicated in each case, contains no unsaturation, and is bound to the rest of the molecule by means of a single bond, for example, methyl, ethyl, n-propyl, i-propyl, n-butyl, t-butyl, n-pentyl, etc.

“Aryl” refers to radicals having individual and multiple aromatic rings, including radicals having multiple rings containing separated and/or condensed aryl groups. Typical aryl groups contain from 1 to 3 separated or condensed rings and from 6 to about 18 carbon ring atoms, such as phenyl, naphthyl, indenyl, phenanthryl or anthracyl radical.

“Halogen” refers to bromine, chlorine, iodine or fluorine.

In the context of the present invention, the salt of the cannabinoid agonist is a pharmaceutically acceptable salt. In the context of the present invention, “pharmaceutically acceptable salt” is understood to be any salt that is physiologically tolerated (usually meaning that it is not toxic, particularly as a result of the counterion) when it is used in a suitable manner for a treatment applied or used, particularly in human beings and/or mammals. The term “salt” must be understood as any form of a cannabinoid agonist according to this invention in which said compound is in ionic form, for example anionic or cationic, and coupled to a counterion, for example and respectively to a cation or anion. Preferably, in the salt the cannabinoid agonist according to the invention is protonated, for example in the nitrogen or oxygen, and the counterion is an anion. Examples of salts of this type are the formed from a cannabinoid agonist of the invention, and hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The term “solvate” according to this invention must be understood to mean any form of the cannabinoid agonist according to the invention in which said compound is bound by means of a non-covalent bond to another molecule (usually a polar solvent), especially including hydrates and alcoholates, such as for example, methanolate. A preferred solvate is hydrate.

The cannabinoid agonists of the invention, or their salts or solvates, preferably have a pharmaceutically acceptable level of purity, excluding normal pharmaceutical additives such as diluents and carriers. The levels of purity are preferably greater than 50%, more preferably greater than 70%, even more preferably greater than 90%. In a preferred embodiment, they are greater than 95%.

As they are used herein, the terms “treating”, “treatment” and derivatives thereof include the reversion, relief, or control of irreversible-type dementia, and more particularly of the cognitive effects associated with dementia, and even more particularly of the learning and/or memory impairment associated with the dementia.

As they are used herein, the terms “prevention”, “preventing” and derivatives thereof refer to the capacity of the cannabinoid agonists of the invention for impeding, minimizing or hindering the onset of dementia in patients whose clinical picture is usually associated with the onset of said dementia.

The present invention furthermore provides medicinal products or pharmaceutical compositions comprising a cannabinoid agonist of this invention as the drug substance, together with a pharmaceutically acceptable excipient, for use in the prevention or treatment of an irreversible-type dementia.

The term “excipient” refers to components of a pharmacological compound other than the drug substance(s) (definition obtained from the European Medicines Agency, EMA). They preferably include a “carrier, adjuvants and/or vehicle”. Carriers are forms in which substances are incorporated to improve drug administration and efficacy. Pharmacological carriers are used in drug delivery systems, such as controlled release technology to extend pharmacological actions in vivo, reduce drug metabolism and reduce drug toxicity. Carriers are also used in designs to increase the efficacy of drug delivery to the targeted pharmacological sites of action. The adjuvant is a substance added to a pharmacological product that affects the action of the drug substance in a predictable manner. The vehicle is an excipient or a substance, preferably one that has no therapeutic action, which is used as a means to provide volume for the administration of medicinal products. Such pharmaceutical carriers, adjuvants or vehicles may be sterile liquids, such as water and oils, including those of a petroleum, animal, plant or synthetic origin, such as peanut oil, soybean oil, mineral oil, sesame oil and the like, excipients, disintegrating agents, wetting agents or diluents. Suitable pharmaceutical carriers are described in “Remington's Pharmaceutical Sciences” by E. W. Martin. The selection of these excipients and the amount to be used will depend how the pharmaceutical composition is applied.

The pharmaceutical composition according to the present invention can be in any suitable form for the application in human beings and/or animals, preferably human beings, including infants, children and adults, and can be produced by means of conventional methods known by those skilled in the art, for example those described or mentioned in the Spanish and US Pharmacopoeias and similar reference texts. Common dosage form examples are solid dosage forms (tablets, pills, capsules, etc.) or liquid dosage forms (solutions, suspensions or emulsions).

Administration of the compounds of the present invention can be intraperitoneal, intramuscular, intra-articular, intravenous, intra-arterial, intravesical, intraosseous, intracavernous, pulmonary, buccal, sublingual, ocular, intravitreal, intranasal, percutaneous, rectal, vaginal, oral, epidural, intrathecal, intraventricular, intracerebral, intracerebroventricular, intracisternal, intraspinal, perispinal, intracranial, administration by means of needles or catheters with or without pump devices, topical administration, particularly dermal, transdermal or subcutaneous, or other routes for the application thereof.

In one embodiment, the administration is oral, intravenous, intraperitoneal, intracerebral, or intracerebroventricular. In another embodiment, the administration is intravenous, intraperitoneal, intracerebral, or intracerebroventricular. In a preferred embodiment, the administration is intraperitoneal, intracerebral, or intracerebroventricular. More preferably, the administration is intraperitoneal.

In one embodiment, the cannabinoid agonist is administered together with ingredients that increase its solubility, for example organic solvents such as dimethylsulfoxide, propylene glycol, polyethylene glycol, ethanol, glycerol, polyethylene glycol ricinoleate (Cremophor) or polysorbates, preferably Cremophor and/or dimethylsulfoxide, even more preferably Cremophor and dimethylsulfoxide at a ratio of between 2:1 to 1:2, more preferably at a ratio of 1:1. In one embodiment, the cannabinoid agonist is administered in a saline solution comprising at least one ingredient that increases solubility of the cannabinoid agonist selected from those mentioned above. Preferably, the ratio of saline solution:solubilizing ingredients is from 2:2 to 2:30, preferably 2:18.

In the context of the present invention, use of the cannabinoid agonist is understood to be in therapeutically effective amounts. The physician will determine the most suitable dosage of the cannabinoid compounds and this will vary with the dosage form and the particular compound that is chosen, and it will furthermore vary with the patient undergoing treatment, the age of the patient, the type of disease or condition that it being treated. When the composition is administered orally, larger amounts of the active agent will be required to produce the same effect as a smaller amount that is administered parenterally. The compounds are useful in the same way as comparable therapeutic agents are and the dosage level is of the same order of magnitude as that which is generally used with these other therapeutic agents.

In one embodiment, the cannabinoid agonist is administered one or more times a day, for example 1, 2, 3 or 4 times a day. In a particular embodiment is administered once a day.

In one embodiment, the administration is carried out before a learning or memorization process, for example between 3 hours and 10 minutes before the learning or memorization process. In a specific embodiment, the administration is carried out prior to a learning process. In another embodiment, the administration is carried out prior to a memorization process.

In one embodiment, the cannabinoid agonist is administered in one dose or in total daily doses in the range of between 0.001 μg/kg and 30 mg/kg, preferably between 0.001 mg/kg and 10 mg/kg, more preferably between 0.01 mg/kg and 10 mg/kg, preferably between 0.01 mg/kg and 5 mg/kg, preferably between 0.01 mg/kg and 1 mg/kg, more preferably between 0.1 mg/kg and 10 mg/kg, preferably between 0.1 mg/kg and 5 mg/kg, preferably between 0.1 mg/kg and 1 mg/kg, and especially preferably between 0.45 and 0.55 mg/kg, and more preferably between 0.49 and 0.51 mg/kg. In another embodiment, the cannabinoid agonist is administered in one dose or in total daily doses of 0.4 to 10 mg/kg, preferably 0.4 to 5 mg/kg, more preferably 0.4 to 1 mg/kg. In the most specific embodiment, the cannabinoid agonist is administered in one dose or in total daily doses of 0.5 mg/kg. Intraperitoneal administration is particularly preferred at these doses.

In one embodiment, the cannabinoid agonist is administered in one dose or in total daily doses of between 0.001 mg/kg and less than 5 mg/kg, preferably between 0.01 mg/kg and less than 5 mg/kg, preferably between 0.1 mg/kg and less than 5 mg/kg, and especially preferably between 0.45 and less than 5 mg/kg.

Intraperitoneal administration is particularly preferred at these doses.

In one embodiment, the cannabinoid agonist is administered in one dose or in total daily doses of between 0.001 mg/kg and less than 1 mg/kg, preferably between 0.01 mg/kg and less than 1 mg/kg, preferably between 0.1 mg/kg and less than 1 mg/kg, and especially preferably between 0.45 and less than 1 mg/kg.

Intraperitoneal administration is particularly preferred at these doses.

In any of the embodiments described above, administration is performed for at least five days. In another embodiment, administration is performed for at least one week. In another embodiment, administration is performed for at least three weeks. In another embodiment, administration is performed for at least one month. In another embodiment, administration is performed for at least three months. In another embodiment, administration is performed for at least six months.

The inventors have found that the beneficial effects of the cannabinoid agonist on cognitive deficits are specifically mediated by the CB₁ receptor.

More than one of the cannabinoid agonists mentioned in this application can be used, or at least another active ingredient against irreversible-type dementia can be used together with the cannabinoid agonist(s) mentioned in this application to provide a combination therapy. The additional cannabinoid agent or the at least another active ingredient can be part of the same composition, or it can be provided as a separate composition for the administration thereof at the same time or at a different time. In one embodiment, the at least another active ingredient against the irreversible-type dementia is selected from: cholinesterase (AChE) inhibitors, for example donepezil, rivastigmine and galantamine; and NMDA glutamate receptor blockers, for example memantine.

In the context of the present invention, dementia is understood as a condition presenting learning and/or memory impairment along with at least one other cognitive deficit, as defined in the fourth edition of the Diagnostic and Statistical Manual of Mental Disorders (DSM-IV) of the American Psychiatric Association, the content of which is incorporated herein by reference.

In a preferred embodiment, dementia is a condition presenting learning and/or memory impairment along with at least one other cognitive deficit that is selected from aphasia, apraxia, agnosia and an executive function disorder. In the context of the present invention, aphasia, apraxia, agnosia and an executive function disorder are understood according to the meaning provided in the fourth edition of the Diagnostic and Statistical Manual of Mental Disorders (DSM-IV) of the American Psychiatric Association.

Dementias treated according to the present invention are irreversible-type dementias, i.e., these exclude dementias produced by causes which can be eliminated, such as dementias due to substance abuse, for example, such as alcohol abuse, dementias due to dietary deficiencies such as a vitamin deficiency, due to head traumas, or due to fever or depression. In a preferred embodiment, irreversible-type dementias are dementias due to a medical condition. Preferably, they are irreversible-type dementias due to a neurodegenerative disease.

In one embodiment, the irreversible-type dementia is selected from: Alzheimer-type dementia, vascular dementia, dementia due to Parkinson's disease, dementia due to Huntington's disease, dementia due to Pick's disease, dementia due to Creutzfeldt-Jakob disease, dementia due to HIV disease, and a combination thereof.

In one embodiment, the irreversible-type dementia is not vascular dementia, and in a more particular embodiment it is not vascular dementia due to cerebral apoplexy or craniocerebral trauma.

In one embodiment, the irreversible-type dementia is selected from: Alzheimer-type dementia, dementia due to Parkinson's disease, dementia due to Huntington's disease, dementia due to Pick's disease, dementia due to Creutzfeldt-Jakob disease, dementia due to HIV disease, or a combination thereof.

In a preferred embodiment, the irreversible-type dementia is Alzheimer-type dementia or dementia due to Parkinson's disease. In a more preferred embodiment, the irreversible-type dementia is Alzheimer-type dementia.

In one embodiment, the Alzheimer-type dementia is early-onset, more particularly uncomplicated early-onset Alzheimer-type dementia, with delusions or a depressed mood; or late-onset, more particularly uncomplicated late-onset Alzheimer-type dementia with delusions or a depressed mood.

In one embodiment, the vascular dementia is uncomplicated vascular dementia, with delusions or a depressed mood.

In the context of the present invention, each of the aforementioned dementias is understood according to the meaning provided in the fourth edition of the Diagnostic and Statistical Manual of Mental Disorders (DSM-IV) of the American Psychiatric Association.

In one embodiment, the use of the cannabinoid agonist is for the prevention or treatment of a cognitive deficit associated with irreversible-type dementia, wherein the cognitive deficit is chosen from: learning and/or memory impairment, aphasia, apraxia, agnosia and an executive function disorder. In a preferred embodiment, the cognitive deficit is learning and/or memory impairment. In one embodiment, the cognitive deficit is a memory impairment, preferably spatial memory impairment. In an even more preferred embodiment, the cognitive deficit is a learning impairment.

In one embodiment, the use of the cannabinoid agonist is for the prevention or treatment of a dementia due to a lesion of the basal forebrain, preferably a cholinergic lesion of the basal forebrain. Cholinergic lesion is understood to be a lesion that impedes cholinergic innervation of the areas responsible for cognitive processes.

In the most preferred embodiment, in any of the embodiments described above, the cannabinoid agent is a compound of formula (Ia) or (Ib) or a salt or solvate thereof.

### EXAMPLES

### Example 1A: Use of Cannabinoid Agonist in an Animal Model of Alzheimer-Type Dementia (1 Round of Training Sessions)

The animal model used is the lesion of cholinergic cells of nucleus basalis magnocellularis in rats to cause memory loss that is sustained over time. It is a recognized model of irreversible-type dementia, specifically of irreversible Alzheimer-type dementia.

More specifically, a bilateral lesion was generated in the nucleus basalis magnocellularis in the brain of male Sprague Dawley rats (male 150-200 g) by means of stereotaxic coordinates (bregma −1.5 mm anteroposterior, ±3 mm laterodorsal and −8 mm ventral from the surface of the cranium) by injecting toxin 192 IgG-saporin (Merck-Millipore. Ref:MAB390) (SAP, n=30) (135 ng/μl/hemisphere; 0.25 μl/min) dissolved in artificial cerebrospinal fluid (CSF, n=28) (148 mM NaCl, 2.7 mM KCl, 0.85 MgCl₂.6H₂O, 1.2 mM CaCl₂.2H₂O; pH 7.4 adjusted with 1 mM K₂HPO₄) and the same method was carried out for the lesion control group but in this case only CSF (1 μl/hemisphere) was injected. Finally, as control of the latter, another group of animals was used which only received the injection of the needle and no compound was administered (SHAM, n=7). To stabilize the lesion process, the animals were not handled for one week. The animals were subjected to an operation on day 0; they were left a week to rest and then training and treatment started on day 8; they received a dose of 0.5 mg/kg of WIN55,212-2 (Tocris. Ref. 1038), one hour before each training session and another dose on the day of the test. The compound is dissolved in DMSO:Cremophor:saline according to ratio [1:1:18]. Each animal receives a single daily intraperitoneal administration of compound WIN55,212-2 dissolved in a vehicle in a volume of 5 ml/kg, amounting to a total of 0.5 mg/kg of WIN55,212-2 a day.

The test used to evaluate the cognitive deficit of these models was the Barnes maze, which measures spatial memory. It is a circular maze with 20 holes on the periphery and only one of them has an escape box. When the animal finds this box, it is returned to its cage. This method is performed four times a day for four days (16 training sessions). On the fifth day, the hole leading to the escape box is covered and the animal explores the maze for three minutes for the purpose of finding this hole (test). The time in the target quadrant will be evaluated in the test. This parameter indicates if the animal accurately remembers where the escape hole is located because it will spend more time in the quadrant containing said hole (target quadrant). This parameter was analyzed by means of SMART 3.0 video-tracking software. Compared to other types of behavioral tests that evaluate memory in rodents, this test has the advantage that there is no aversive stimulus with which effects relating to fear and anxiety may interfere, which thereby prevents confusing anxiolytic effects with cognitive enhancing effects.

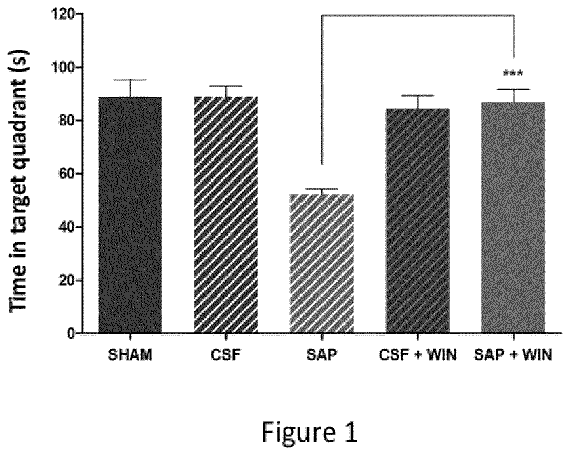

The results clearly and surprisingly showed that the lesioned group treated with the cannabinoid agonist (SAP+WIN, n=12) had no memory deficits, and furthermore the same compound had no effect on the CSF group (CSF+WIN, n=12) (see FIG. 1).

It is therefore unexpectedly proven that at the dosage levels used, which are levels at which the cannabinoid agonist has no type of effect on animals in which Alzheimer-type dementia was not induced, the cannabinoid agonist is indeed capable of counteracting the cognitive deficits of the animals in which damage to the learning and memory capacity, which can be extrapolated to Alzheimer-type dementia, was indeed induced.

### Example 1B: Use of a Cannabinoid Agonist in an Animal Model of Alzheimer-Type Dementia (2 Rounds of Training Sessions)

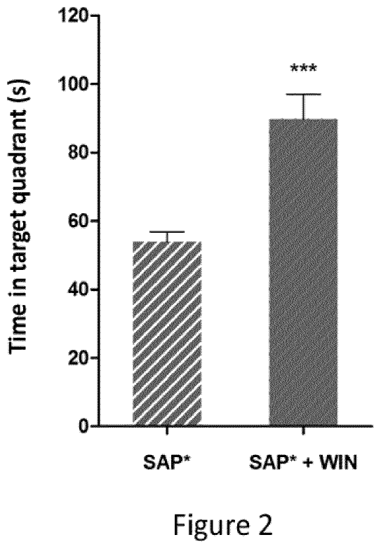

In this case, the method was carried out like in the preceding example except training sessions, in this case without treatment, started on day 8. The results show that as occurred in earlier experiments, the saporin group (SAP*, n=20) shows a cognitive deficit in the Barnes test, but they are left to rest for a week, and new training sessions start on day 18, changing the escape hole, and they are administered on the four days of training and on the day of the test a dose comprising 0.5 mg/kg of WIN55,212-2 an hour before subjecting the animal to the test (SAP*+WIN). The results show that the same animals that were previously unable to remember the escape hole are capable of remembering it in this case with the aid of the treatment (see FIG. 2).

### Example 2: Use of Cannabinoid Agonist and Antagonist Co-Administration in an Animal Model of Alzheimer-Type Dementia to Check if the Effect Observed is Mediated by CB1 Receptor Activation

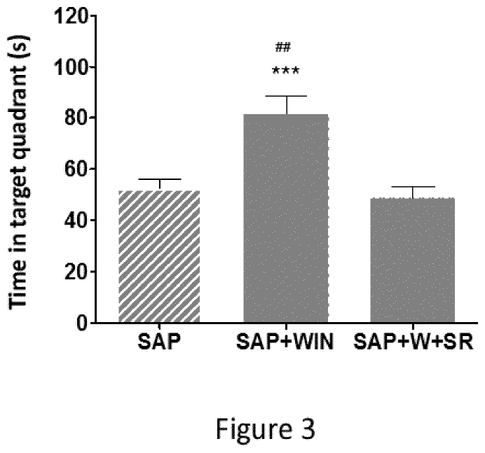

In this case, the treatment regimen was the same as in the method explained in example 1A. Together with the dose of 0.5 mg/kg of WIN55,212-2, 0.5 mg/kg of SR141716A (SAP+W+SR, n=9), a selective CB1 receptor antagonist, was administered. As observed in example 1A, administration of the toxin (i.c.) produces a cognitive deficit reflected in the Barnes maze test, where the animal spends less time in the target quadrant. Furthermore, treatment with the cannabinoid agonist at a dose of 0.5 mg/kg achieves that the injured rat spends the most amount of time in the target quadrant. Finally, agonist and antagonist co-administration at the same dose (0.5 mg/kg) achieves reversing the effect produced by treatment with WIN55,212-2 alone (see FIG. 3).

### Example 3: Use of Cannabinoid Agonist at a High Dose (3 mg/kg) in an Animal Model of Alzheimer-Type Dementia

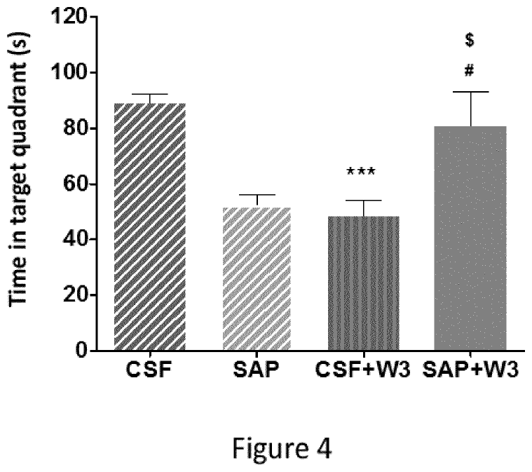

Once again, the same drug administration scheme as the one explained in example 1A was carried out for this experiment. The main difference is the increase in the dose of the compound WIN55,212-2 from 0.5 mg/kg to 3 mg/kg. The obtained results show that by increasing the dose, cannabinoid agonism is detrimental for memory in a control rat given that it spends less time in the target quadrant than a corresponding rat receiving no cannabinoid treatment. In contrast, the injured rats behave like a control rat, spending more time in the target quadrant but with effects comparable to those obtained at lower doses (0.5 mg/kg) (see FIG. 4).

1. A method of prevention or treatment of an irreversible-type dementia due to a cholinergic lesion of the basal forebrain, the method comprising a) providing a subject in need of prevention or treatment of such an irreversible-type dementia; and b) administering to said subject a compound of formula (Ia) or (Ib) 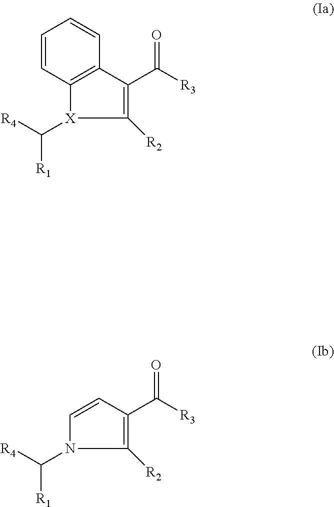
wherein
X is N or CH;
R₁ is selected from
C₂-C₆ alkyl;
a piperidinyl, pyrrolidinyl or oxazinanyl group that is unsubstituted or substituted with methyl at its nitrogen atom; and
a —CH₂-morpholinyl, —CH₂-thiomorpholinyl or —CH₂— piperidinyl group;
R₂ is selected from H, C₁-C₆ alkyl, halogen, —CN and —OH;
R₃ is selected from unsubstituted aryl and aryl substituted at one or more of its available positions with C₁-C₆ alkyl, —O—C₁-C₆ alkyl, halogen, —CN, —NO₂ or —OH;
R₄ is H, and in the compound of formula (Ia) it can also be a —CH₂—Y group, wherein Y is O, S, or NR₅, wherein Y is bound directly to the 7-position of the indole or indene ring, and wherein R₅ is H or C₁-C₆ alkyl;
and wherein in the compound of formula (Ia), the phenyl portion of the indole ring (X is N) or indene ring (X is CH) is unsubstituted or substituted at one or more of its available positions with C₁-C₆ alkyl, —O—C₁-C₆ alkyl or halogen;
or a salt or solvate thereof;
wherein the compound is administered at a dose of between 0.001 mg/kg and less than 5 mg/kg.

2. The method according to claim 1, wherein the compound is a compound of formula (Ia).

3. The method according to claim 1, wherein X is N.

4. The method according to claim 1, wherein R₁ is a —CH₂-morpholinyl, —CH₂-thiomorpholinyl or —CH₂-piperidinyl group.

5. The method according to claim 1, wherein R₂ is H or C₁-C₆ alkyl.

6. The method according to claim 1, wherein R₃ is naphthyl.

7. The method according to claim 1, wherein R₄ is a —CH₂—Y group, wherein Y is O and is bound directly to the 7-position of the indole or indene ring.

8. The method according to claim 1, wherein the compound is 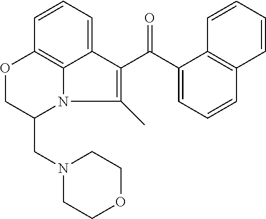 [SMILES: Cc1c(C(=O)c2cccc3ccccc23)c2cccc3c2n1C(CN1CCOCC1)CO3]
or a salt or solvate thereof.

9. The method according to claim 1, wherein the compound is 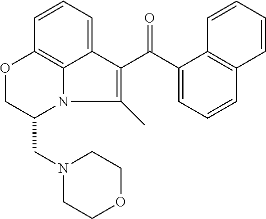 [SMILES: Cc1c(C(=O)c2cccc3ccccc23)c2cccc3c2n1[C@H](CN1CCOCC1)CO3]
or a salt or solvate thereof.

10. The method according to claim 1, wherein the compound of formula (Ia) or (Ib) or its salt or solvate is administered at a dose of between 0.1 mg/kg and less than 5 mg/kg.

11. The method according to claim 1, wherein the administered dose is a daily dose.

12. The method according to claim 1, wherein the compound of formula (Ia) or (Ib) or its salt or solvate is administered intraperitoneally.

13. The method according to claim 1, wherein the irreversible-type dementia is selected from the group consisting of: Alzheimer-type dementia, vascular dementia, dementia due to Parkinson's disease, dementia due to Huntington's disease, dementia due to Pick's disease, dementia due to Creutzfeldt-Jakob disease, dementia due to HIV disease, and a combination thereof.

14. The method according to claim 1, wherein the irreversible-type dementia is Alzheimer-type dementia.

15. The method according to claim 1, wherein the compound of formula (Ia) or (Ib) is used together with at least one active ingredient selected from a cholinesterase inhibitor and an NMDA glutamate receptor blocker.
